(19) United States
(12) Patent Application Publication
(10) Pub. No.: US 2021/0128910 A1
(43) Pub. Date: May 6, 2021

(54) ELECTROPORATION PROBE

(71) Applicant: MIRAI MEDICAL LIMITED, Oranmore (IE)

(72) Inventors: Declan SODEN, Clonakilty (IE); Colin FORDE, Kinvara (IE)

(73) Assignee: MIRAI MEDICAL LIMITED, Oranmore (IE)

(21) Appl. No.: 17/086,498

(22) Filed: Nov. 2, 2020

Related U.S. Application Data
(60) Provisional application No. 62/931,397, filed on Nov. 6, 2019.

(51) Int. Cl. A61N 1/32; A61N 1/04; A61M 37/00

(52) U.S. Cl. CPC A61N 1/327; A61M 2037/0007; A61M 37/00; A61N 1/0412

(57) ABSTRACT

An electroporation probe has a housing supporting needles electrically linked to a coupler for an electrical drive. A cap has apertures in a distal face and aligned with the needles. The cap is movable longitudinally against a spring bias relative to the needles to allow insertion by the needles in use as the cap is pressed against patient tissue. There is a limit feature for abutting a limiter setting maximum extent of longitudinal movement relative to the housing. This allows the probe to be presented by the physician without the needles protruding and for them to be inserted up to the user-set maximum extent with maintenance of spacings due to the guiding action of the cap pressed against the tissue surface.

Electroporation is a medical and molecular biology technique in which an electric field is applied in order to increase the permeability of the cell membrane, allowing chemicals, drugs, or DNA to be introduced into the cell. Electroporation has a number of possible fields of application, and can be used both reversibly and irreversibly. In a typical electroporation process, short and intense electric pulses are generated to transiently permeabilize the cell membrane.

In reversible electroporation, which may be used in relation to for example chemotherapy, the electric field is below an electric field threshold and allows the cell membrane to repair after the treatment. Reversible electroporation may involve allowing a molecule, such as a drug or gene, into a cell or molecule that is normally not permeable for this substance without inducing cell death. The electric field threshold for a cell is individual for the cell. In irreversible electroporation the electric field is greater than the electric field threshold, which creates permanent nanopores in the cell membrane, disrupting the cellular homeostasis and consequently the cell dies by a combination of apoptosis and necrosis.

It is generally understood that electroporation of the cell provides a beneficial impact in a variety of biological and microbiological applications that insert molecules through the cell membrane. Organic and inorganic transfers into the cells benefit from the application of high voltage electrical charges to the recipient cell membrane, in advance of the treatment.

In many cases electroporation needles of a probe carry the drive current provided by a drive circuit, and in use penetrate the skin to a desired extent according to the clinical requirements. The needles are preferably as thin as possible in order to reduce risk of patient trauma during physical insertion. However, thinner needles have a greater risk of buckling or laterally moving, thereby presenting the risk of electrical shorting or excessive energy delivered to the patient. This risk is particularly acute if the needles need to penetrate to a depth of greater than about 5 mm, and/or if required to be used many times for a treatment over a large area. The distance between the electrodes is also essential for effective treatment deliver. If the needles bend closer together/further apart it can lead to less than effective treatment and/or damage to the patient.

EP2693959 (IGEA S.p.A describes a hand-held adjustable device with an end portion with a grip, needles on the end portion, and a cup-shaped body which axially slides relative to the end portion. There is a plurality of stable axial portions between the cup-shaped body and the end portion, achieved by a stop element extending radially from the end portion and engaging transversal seats defining the stable axial positions. Also, WO2017/117508 (Inovio) also describes an electroporation device.

In an outpatient setting, exposed needles can make the patient experience less optimal due to fear of needles and also increase the risk of needle stick injuries for the user.

The invention addresses these problems.

### SUMMARY

We describe an electroporation probe comprising:
a housing supporting needles electrically linked to a coupler for an electrical drive,
a cap having apertures aligned with the needles and a distal face for engagement with patient tissue;
the cap and the needles being movable longitudinally relative to each other against a spring bias to allow penetration by the needles with movement through the cap apertures, and
a user-settable limiter which is movable relative to the housing to set a maximum extent of mutual longitudinal movement of the needles and the cap face;
wherein the cap is movable longitudinally against a spring bias relative to the needles, and the needles are fixed relative to the housing, to allow penetration by the needles in use as the cap is pressed against patient tissue, up to a maximum extent set by axial position of the user-settable limiter relative to the housing.

Preferably, there are at least two needles. Preferably, there are in the range of 4 to 12 needles.

Preferably, the cap comprises a flange for engaging the limiter.

Preferably, the limiter comprises a user button which is movable relative to the housing to set said mutual axial movement limits. Preferably, wherein the limiter comprises a user button which is movable relative to the housing to set said mutual axial movement limits; and wherein the limiter is rotatable circumferentially relative to the housing to a neutral position and is slidable longitudinally relative to the housing to a desired limiting position, and is rotatable circumferentially relative to the housing to a different set limiting position.

In one example, the cap is biased distally by a spring acting between the housing and the cap. In one example, the cap narrows leading to its distal face. In one example, the cap has a frusto-conical shape leading to the distal face.

Preferably, the needles are mounted by physical engagement in individual sockets each of which grips a needle base.

In one example, the needles are mounted by physical engagement in individual sockets each of which grips a needle base; and wherein the sockets are mounted in an array on a distally-facing face of the needle base, and a rear face of said base is electrically connected to an electrical drive conductor. In one example, the needle base comprises a printed circuit board.

In One Example:
the needles are mounted by physical engagement in individual sockets each of which grips a needle base; and the sockets are mounted in an array on a distally-facing face of the needle base, and a rear face of said base is electrically connected to an electrical drive conductor, and
the needle base comprises a printed circuit board.

In one example, there is a plurality of caps, each associated with a subset of the needles and each being individually movable relative to its associated needles or at least some of the needles are movable relative to their associated caps.

In one example, the probe further comprises visualisation actuator ring having slider buttons which allow the user to pull back the cap to a maximum extent against action of a spring for temporary retraction of the cap before contact with the patient.

In one example, the limiter comprises a user button which is movable relative to the housing to set said mutual axial movement limits; and wherein the limiter is rotatable circumferentially relative to the housing to a neutral position and is slidable longitudinally relative to the housing to a desired limiting position, and is rotatable circumferentially relative to the housing to a different set limiting position; and wherein the user-settable limiter comprises a slider mounted to slide longitudinally within the housing, and a limiter ring within the slider, the limiter ring being free to move longitudinally with the slider and to rotate relative to the slider within limits set by the slider.

In one example, the limiter comprises a user button which is movable relative to the housing to set said mutual axial movement limits; and wherein the limiter is rotatable circumferentially relative to the housing to a neutral position and is slidable longitudinally relative to the housing to a desired limiting position, and is rotatable circumferentially relative to the housing to a different set limiting position; and wherein the user-settable limiter comprises a slider mounted to slide longitudinally within the housing, and a limiter ring within the slider, the limiter ring being free to move longitudinally with the slider and to rotate relative to the slider within limits set by the slider, and wherein the limiter ring and the slider comprises features which abut to provide a haptic feedback when the button is moved to a position

We also describe an electroporation apparatus comprising an electrical drive for generating electroporation pulses, a probe, and a coupler for coupling the probe to the electrical drive, wherein the probe comprises a housing supporting needles electrically linked to a coupler for an electrical drive,
a cap having apertures aligned with the needles and a distal face for engagement with patient tissue;
the cap and the needles being movable longitudinally relative to each other against a spring bias to allow penetration by the needles with movement through the cap apertures, and
a user-settable limiter which is movable relative to the housing to set a maximum extent of mutual longitudinal movement of the needles and the cap face;
wherein the cap is movable longitudinally against a spring bias relative to the needles, and the needles are fixed relative to the housing, to allow penetration by the needles in use as the cap is pressed against patient tissue, up to a maximum extent set by axial position of the user-settable limiter relative to the housing.

We also describe a method of operation of an electroporation probe comprising:
a housing supporting needles electrically linked to a coupler for an electrical drive,
a cap having apertures aligned with the needles and a distal face for engagement with patient tissue;
the cap and the needles being movable longitudinally relative to each other against a spring bias to allow penetration by the needles with movement through the cap apertures, and
a user-settable limiter which is movable relative to the housing to set a maximum extent of mutual longitudinal movement of the needles and the cap face;
wherein the cap is movable longitudinally against a spring bias relative to the needles, and the needles are fixed relative to the housing, to allow penetration by the needles in use as the cap is pressed against patient tissue, up to a maximum extent set by axial position of the user-settable limiter relative to the housing;
wherein the method comprises steps of:
adjusting the user-settable limit by moving the limiter relative to the housing to select one of a plurality of limit settings for position of the cap relative to the housing,
pressing the cap against patient tissue with sufficient force to cause the cap to retract towards the housing and the needles of be exposed up to a maximum set by said limit.

In one example, the probe further comprises comprising a visualisation actuator ring having diametrically opposed slider buttons which allow the user to pull back the cap to a maximum extent against action of a spring for temporary retraction of the cap before contact with the patient, and the method comprises initially temporarily pulling back the cap against action of said spring before contact of the cap with the patient tissue.

We Describe an Electroporation Probe Comprising:
a housing supporting needles electrically linked to a coupler for an electrical drive,
a cap having apertures aligned with the needles and a distal face for engagement with patient tissue;
the cap and the needles being movable longitudinally relative to each other against a spring bias to allow penetration by the needles with movement through the cap apertures, and
a user-settable limiter which is movable relative to the housing to set a maximum extent of mutual longitudinal movement of the needles and the cap face.

Preferably, the cap is movable longitudinally against a spring bias relative to the needles, and the needles are fixed relative to the housing, to allow penetration by the needles in use as the cap is pressed against patient tissue, up to a maximum extent set by axial position of the limiter relative to the housing.

Optionally, there are at least two needles, preferably in the range of 4 to 12 needles. Optionally, the cap comprises a flange for engaging the limiter. Optionally, the limiter comprises a user button which is movable relative to the housing to set said mutual axial movement limits.

Optionally, the limiter is rotatable circumferentially relative to the housing to a neutral position and is slidable longitudinally relative to the housing to a desired limiting position, and is rotatable circumferentially relative to the housing to a different set limiting position. Optionally, the cap is biased distally by a spring acting between the housing and the cap, or the needles are biased proximally by a spring acting between the housing and the needles. Optionally, the cap narrows leading to its distal face. Optionally, the cap has a frusto-conical shape leading to the distal face.

Optionally, the needles are mounted by physical engagement in individual sockets each of which grips a needle base. Preferably, the sockets are mounted in an array on a distally-facing face of the needle base, and a rear face of said base is electrically connected to an electrical drive conductor. Optionally, the needle base comprises a printed circuit board.

Optionally, there is a plurality of caps, each associated with a subset of the needles and each being individually movable relative to its associated needles or at least some of the needles are movable relative to their associated caps.

We also describe an electroporation apparatus comprising an electrical drive for generating electroporation pulses and the probe head as described herein in any example, and a coupler for coupling the probe head to the drive.

### DETAILED DESCRIPTION OF THE INVENTION

The invention will be more clearly understood from the following description of some embodiments thereof, given by way of example only with reference to the accompanying drawings in which:

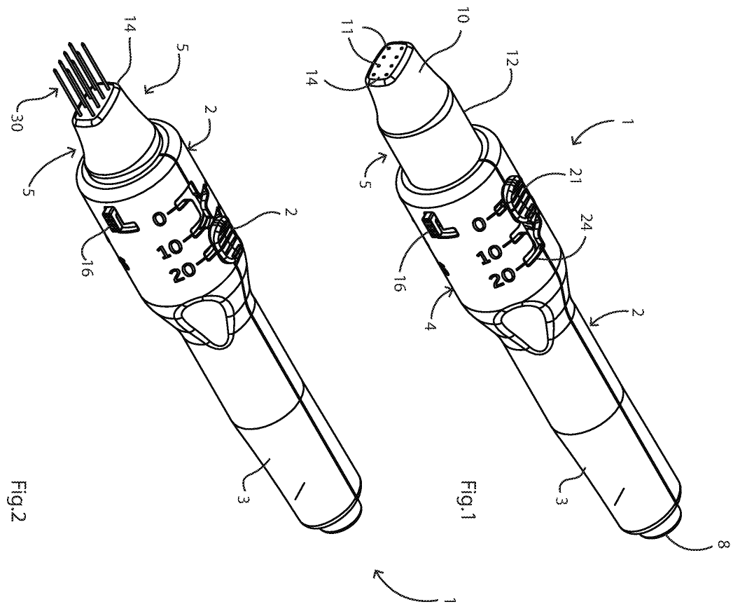

FIGS. 1 and 2 are perspective views of an electroporation probe with eight needles, FIG. 1 showing the needles hidden by a cap and FIG. 2 showing them exposed;

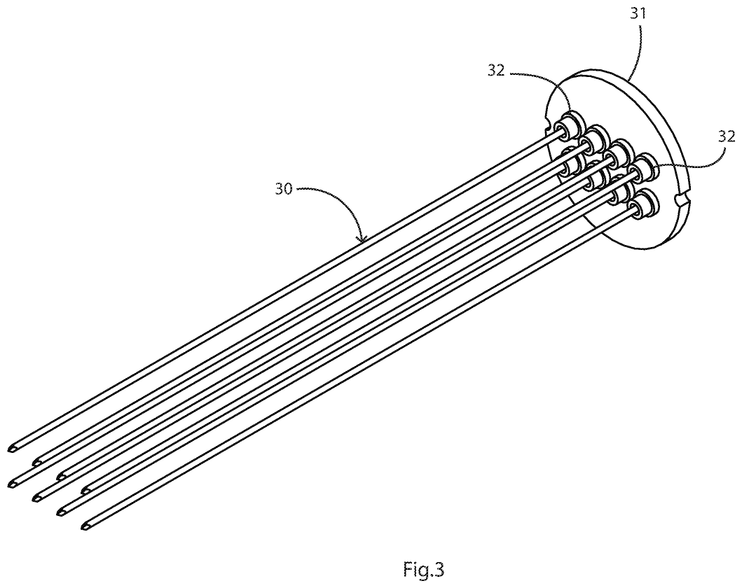

FIG. 3 is a perspective view showing mounting of the needles;

FIG. 4 is an exploded view showing the inside of the probe and the components in more detail; and

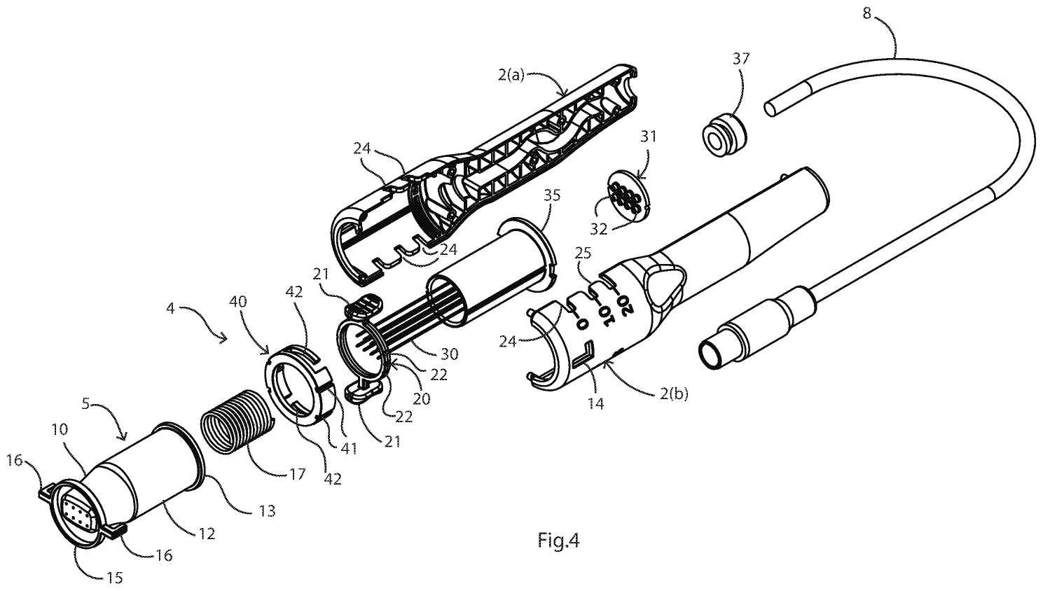

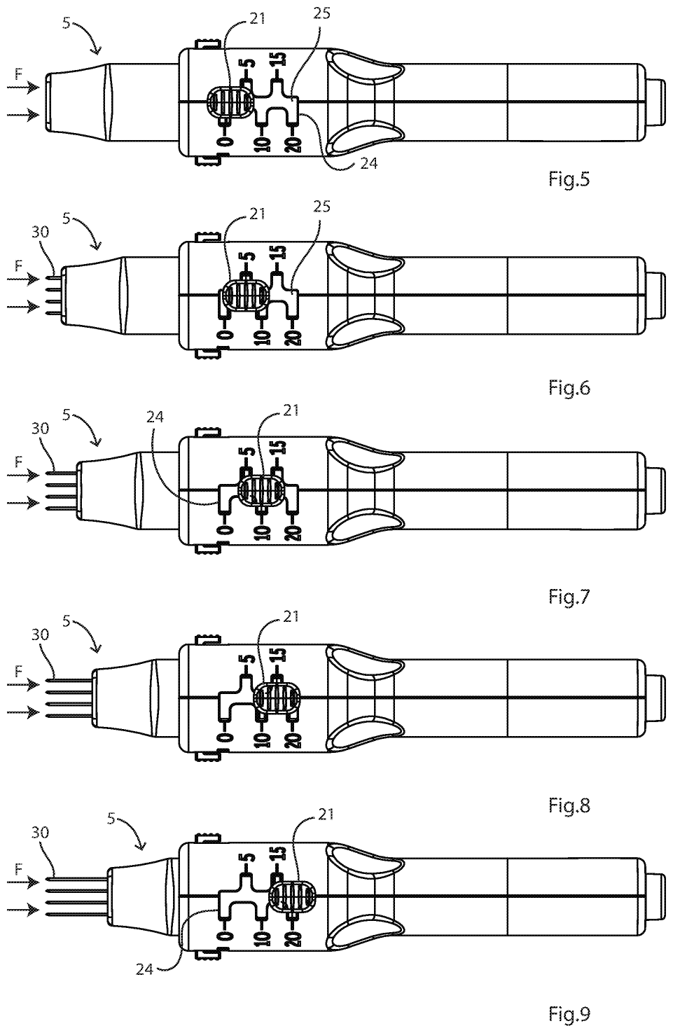

FIGS. 5 to 9 are a series of side views showing a probe with a probe cap at different longitudinal positions thereby revealing or exposing different extents of the needles in use.

In summary, an electroporation probe has a housing supporting needles electrically linked to a coupler for an electrical drive. A cap has apertures in a distal face and aligned with the needles. The cap is movable longitudinally against a spring bias relative to the needles to allow penetration by the needles in use as the cap is pressed against patient tissue. There is a limit feature for abutting an actuator limiter setting maximum extent of longitudinal movement relative to the housing. This allows the probe to be presented by the physician without the needles protruding and for them to be inserted with maintenance of spacings due to the guiding action of the cap pressed against the tissue surface, and insertion into the tissue only to a limited extend which is pressed by the physician. Advantageously, the physician can be certain of the maximum depth even without being able to see the needles before or during insertion into the tissue.

Referring to FIGS. 1 to 4 an electroporation probe 1 has a moulded plastics housing 2 in two parts 2(a) and (b). The housing 2 forms a handle 3 and an actuator 4. At the probe distal end, a cap 5 protrudes from the housing 2, and comprises a distal end face 14 with eight apertures 11 through which needles 30 can extend in use. The cap 5 also has a distal tapered portion 10 leading to the end face 14 and, at its proximal end, a proximal shoulder 12. The cap 5 also has a proximal flange 13 at its proximal end, which is located within the housing 2 and extends radially for engagement with a slider ring 40/limiter ring 20 assembly of the actuator. This is for longitudinal movement of the cap relative to the housing 2 up to a limit set by the actuator 4, as described in more detail below.

The cap 5 is biased distally by a helical spring 17 engaging the cap tapered portion 10 at its distal end and, and at its proximal end, a ledge within a tube 35 which is fixed to the housing 2.

There is a visualisation ring 15 having diametrically opposed slider buttons 16 which allow the user to pull back the cap within a limit set by a slot 14 in the housing to a maximum extent of 5 mm against action of a helical spring 17. This is by the visualisation ring 15 pressing proximally against the cap proximal flange 13. The visualisation ring allows a temporary retraction of the cap 5 during the moments immediately before contact with the patient, typically out of the field of vision of the patient. This helps accurate positioning by the physician.

The actuator 4 has a limiter ring 20 with two opposed radially-extending buttons 21 to allow the physician to set a maximum extent of movement of the cap 5 relative to the housing 2. The buttons 21 can be moved longitudinally in a slot 25 formed between the two housing parts 2(a) and 2(b), and when at a desired location, moved circumferentially into a selected opposed pair of graduated slots 24 in the housing to set a desired limit of 0 mm, 5 mm, 10 mm, 15 mm, or 20 mm for maximum extent of cap movement. The relevant opposed slots 24 hold the limiter ring 20 at the relevant longitudinal position, thus acting as a limit to proximal movement of the flange 13, and hence of the cap 5, during use.

The limiter ring 20 is within a slider ring 40 which has longitudinal grooves 41 on its outside to allow smooth sliding of the ring 40 within the housing, the grooves 41 engaging ridges in the housing 2. Also, the limiter ring 20 can rotate within the slider ring 40, the neck of each button 21 extending radially through a slot 42 in the slider ring 40, the slot 42 limiting the extent of circumferential movement. Moreover, the limiting ring 20 has external grooves 22 which engage dimples inside the slider ring 40, to provide a snap-fitting “clicking” haptic feedback to the user when the buttons 21 are moved into a particular slot 24. The dimples prevent the limiting ring 20 from disengaging from its desired depth setting without user input. Hence, the slider ring 40 slides within the housing 2 in a smooth action by virtue of the longitudinal grooves 41, houses the limiter ring 20, allows controlled rotation of the limiter ring 20 by engagement of the necks of the buttons 21 in the slider circumferential slots 42, and provides haptic feedback for the button positions by virtue of engagement of the limiter ring slots 22 with dimples within the slider ring 40.

The needles 30 are mounted on a needle support 31 (also a printed circuit board (PCB)), in which each needle is mounted to an individual socket 32. The rear face of the needle support 31 is soldered to conductors of a cable 8 from a high voltage drive. The needle support 31 is fixed to the tube 35, in turn fixed to the housing 2, so that the needle position does not change relative to the housing 2. As the PCB (31) has pre-set electrical conductors it ensures that no cross wiring can occur to the needles.

Referring to FIGS. 5 to 9 five cap positions are shown, arising from distal penetration of the needles 30 into the patient's tissue and the cap face resting against the tissue surface, indicated by the arrows F. The physician pushes the probe 1 against the treatment area, and up until contact with the skin the needles 30 are not visible, as shown in FIG. 1. Hence, the patient does not have visibility of the needles 30, thus reducing risk of patient mental trauma and needle stick injury to the user. In some cases, however the physician may temporarily expose up to 5 mm of the needle tips by retracting the slider 15, for improved accuracy of needle positioning if desired. The time duration of needle tip exposure is very limited and is likely to be out of the patient's field of vision.

As the probe is pushed against the treatment area, the cap moves proximally under pressure from the tissue as the needles 30 penetrate the tissue. By the physician setting the position of the limiter buttons 24, the extent of needle penetration is limited to 5 mm, 10 mm, 15 mm, or 20 mm as shown in in FIGS. 6 to 9 respectively. FIG. 6 shows 5 mm penetration, FIG. 7 10 mm, FIG. 8 15 mm, and FIG. 9 20 mm penetrations.

It will be appreciated that the probe avoids the need for the needles to be visible to the patient, who is likely to be only under a local anaesthetic. Also, it acts as a guide for the needles, the apertures 11 providing support, thereby significantly reducing risk of lateral needle movement and buckling, while the physician has full control over the extent of maximum penetration of the needles into the tissue. The needles are a contact fit within the apertures 11 of the cap 5, thereby preventing significant needle lateral movement.

The probe may be used with any type of electroporation, either for cell ablation or permeabilization.

The probe may be used internally if there is a tissue surface exposed for example in the oral cavity or in a surgically-opened area.

The probe may include a stem which may or may not be longer than the handle 3, and the stem may include an endoscope and/or an articulating joint. The articulating joint may also be at the distal end of the device.

Maintaining the needle gap while also controlling the maximum extent of insertion of the needles ensures effective delivery of energy to the tissue and also reduced risk of shorting between the electrodes which may result in tissue overheating and/or damage to the drive generator. Advantageous features which maintain the needle gap include.
a. The needle guide apertures 11 in the cap 5 help to maintain the needle gap at critical times in the procedure, namely, penetration and travel through tissue to tumour for example.
b. The cap and the spring ensure that the needles are supported as close to the needle entry point as possible.
c. Needle holder (35) helps maintain needle gap within the body (3).

The probe also allows for accurate insertion of needles as the inwardly-tapered cap profile 10 allows good visibility and accuracy of needle placement. Also, the needle tip exposure device 15/16 allows for up to 5 mm of the needle tip to be temporarily exposed for better visualisation of the needle entry point.

Also, needle stick injuries are a risk with any needle-based therapy, and in this case the spring-loaded front cap 5 automatically covers the needles when product is not in use.

User contact with needles resulting in shock is avoided because the spring-loaded cap 5 ensures that there is no needle/electrode surface exposed for contacting.

The invention is not limited to the embodiments described but may be varied in construction and detail. For example, the cap may be limited in its longitudinal movement by any other suitable user-actuated limiter, such as rotating and threaded slides which move relative to the housing. Such an arrangement may allow any maximum extent of movement, not just discrete settings.

In other cases, there may be independently-moving front caps for uneven surfaces. For example, each cap houses a separate needle and has own spring so can cater for uneven skin surfaces more efficiently. For example, instead of one cap for the eight needles there is an individual cap for each needle. i.e. eight caps which move independently. This can then adjust to any surface topography

In other examples, the cap is stationary relative to at least some of the housing, and the needles are movable relative to the cap and are preferably spring-loaded. In this case a slider akin to the slider 20/21 is attached to the needles and pushes them forwardly. The user controls depth of needle penetration and once user (for example, thumb) pressure is removed the needles spring back within the front cap.

1. An electroporation probe comprising:
a housing supporting needles electrically linked to a coupler for an electrical drive,
a cap having apertures aligned with the needles and a distal face for engagement with patient tissue;
the cap and the needles being movable longitudinally relative to each other against a spring bias to allow penetration by the needles with movement through the cap apertures, and
a user-settable limiter which is movable relative to the housing to set a maximum extent of mutual longitudinal movement of the needles and the cap face;
wherein the cap is movable longitudinally against a spring bias relative to the needles, and the needles are fixed relative to the housing, to allow penetration by the needles in use as the cap is pressed against patient tissue, up to a maximum extent set by axial position of the user-settable limiter relative to the housing.

2. The electroporation probe as claimed in claim 1, wherein there are at least two needles,

3. The electroporation probe as claimed in claim 1, wherein there are in the range of 4 to 12 needles.

4. The electroporation probe as claimed in claim 1, wherein the cap comprises a flange for engaging the limiter.

5. The electroporation probe as claimed in claim 1, wherein the limiter comprises a user button which is movable relative to the housing to set said mutual axial movement limits.

6. The electroporation probe as claimed in claim 1, wherein the limiter comprises a user button which is movable relative to the housing to set said mutual axial movement limits; and wherein the limiter is rotatable circumferentially relative to the housing to a neutral position and is slidable longitudinally relative to the housing to a desired limiting position, and is rotatable circumferentially relative to the housing to a different set limiting position.

7. The electroporation probe as claimed in claim 1, wherein the cap is biased distally by a spring acting between the housing and the cap.

8. The electroporation probe as claimed in claim 1, wherein the cap narrows leading to its distal face.

9. The electroporation probe as claimed in claim 1, wherein the cap has a frusto-conical shape leading to the distal face.

10. The electroporation probe as claimed in claim 1, wherein the needles are mounted by physical engagement in individual sockets each of which grips a needle base.

11. The electroporation probe as claimed in claim 1, wherein the needles are mounted by physical engagement in individual sockets each of which grips a needle base; and wherein the sockets are mounted in an array on a distally-facing face of the needle base, and a rear face of said base is electrically connected to an electrical drive conductor.

12. The electroporation probe as claimed in claim 1, wherein the needle base comprises a printed circuit board.

13. The electroporation probe as claimed in claim 1, wherein:
the needles are mounted by physical engagement in individual sockets each of which grips a needle base; and the sockets are mounted in an array on a distally-facing face of the needle base, and a rear face of said base is electrically connected to an electrical drive conductor, and
the needle base comprises a printed circuit board.

14. The electroporation probe as claimed in claim 1, wherein there is a plurality of caps, each associated with a subset of the needles and each being individually movable relative to its associated needles or at least some of the needles are movable relative to their associated caps.

15. The electroporation probe as claimed in claim 1, further comprising visualisation actuator ring having slider buttons which allow the user to pull back the cap to a maximum extent against action of a spring for temporary retraction of the cap before contact with the patient.

16. The electroporation probe as claimed in claim 1, wherein the limiter comprises a user button which is movable relative to the housing to set said mutual axial movement limits; and wherein the limiter is rotatable circumferentially relative to the housing to a neutral position and is slidable longitudinally relative to the housing to a desired limiting position, and is rotatable circumferentially relative to the housing to a different set limiting position;
and wherein the user-settable limiter comprises a slider mounted to slide longitudinally within the housing, and a limiter ring within the slider, the limiter ring being free to move longitudinally with the slider and to rotate relative to the slider within limits set by the slider.

17. The electroporation probe as claimed in claim 1, wherein the limiter comprises a user button which is movable relative to the housing to set said mutual axial movement limits; and wherein the limiter is rotatable circumferentially relative to the housing to a neutral position and is slidable longitudinally relative to the housing to a desired limiting position, and is rotatable circumferentially relative to the housing to a different set limiting position;
and wherein the user-settable limiter comprises a slider mounted to slide longitudinally within the housing, and a limiter ring within the slider, the limiter ring being free to move longitudinally with the slider and to rotate relative to the slider within limits set by the slider, and wherein the limiter ring and the slider comprises features which abut to provide a haptic feedback when the button is moved to a position

18. An electroporation apparatus comprising an electrical drive for generating electroporation pulses, a probe, and a coupler for coupling the probe to the electrical drive, wherein the probe comprises a housing supporting needles electrically linked to a coupler for an electrical drive,
a cap having apertures aligned with the needles and a distal face for engagement with patient tissue;
the cap and the needles being movable longitudinally relative to each other against a spring bias to allow penetration by the needles with movement through the cap apertures, and
a user-settable limiter which is movable relative to the housing to set a maximum extent of mutual longitudinal movement of the needles and the cap face;
wherein the cap is movable longitudinally against a spring bias relative to the needles, and the needles are fixed relative to the housing, to allow penetration by the needles in use as the cap is pressed against patient tissue, up to a maximum extent set by axial position of the user-settable limiter relative to the housing.

19. A method of operation of an electroporation probe comprising:
a housing supporting needles electrically linked to a coupler for an electrical drive,
a cap having apertures aligned with the needles and a distal face for engagement with patient tissue;
the cap and the needles being movable longitudinally relative to each other against a spring bias to allow penetration by the needles with movement through the cap apertures, and
a user-settable limiter which is movable relative to the housing to set a maximum extent of mutual longitudinal movement of the needles and the cap face;
wherein the cap is movable longitudinally against a spring bias relative to the needles, and the needles are fixed relative to the housing, to allow penetration by the needles in use as the cap is pressed against patient tissue, up to a maximum extent set by axial position of the user-settable limiter relative to the housing;
wherein the method comprises steps of:
adjusting the user-settable limit by moving the limiter relative to the housing to select one of a plurality of limit settings for position of the cap relative to the housing,
pressing the cap against patient tissue with sufficient force to cause the cap to retract towards the housing and the needles of be exposed up to a maximum set by said limit.

20. The method of claim 18, wherein the probe further comprises comprising a visualisation actuator ring having diametrically opposed slider buttons which allow the user to pull back the cap to a maximum extent against action of a spring for temporary retraction of the cap before contact with the patient, and the method comprises initially temporarily pulling back the cap against action of said spring before contact of the cap with the patient tissue.
